(19) United States
(12) Patent Application Publication
(10) Pub. No.: US 2008/0206328 A1
(43) Pub. Date: Aug. 28, 2008

(54) Hypocholesterolemic Compositions Comprising a Statin and an Antiflatulent Agent

(75) Inventors: Marta Guerrero, Barcelona (ES); Anna Orriols, Manresa (ES); Manuel M. Raga, Bacelona (ES); Antonio Guglietta, Barcelona (ES)

Correspondence Address: BIRCH STEWART KOLASCH & BIRCH, PO BOX 747, FALLS CHURCH, VA, 22040-0747, US

(73) Assignee: Ferrer International, S.A., Barcelona (ES)

(21) Appl. No.: 10/588,377

(22) PCT Filed: Feb. 2, 2005
(86) PCT No.: PCT/EP05/01038

§ 371 Date: Feb. 7, 2007

(30) Foreign Application Priority Data
Feb. 3, 2004 (EP) 04002317.8

(51) Int. Cl. A61K 9/28; A61K 31/40; A61K 31/351; A61K 33/00

(52) U.S. Cl. 424/465; 514/423; 514/460; 424/600; 424/474

(57) ABSTRACT

The present invention relates to hypocholesterolemic compositions comprising statins plus antiflatulent agents. In particular the compositions of the present invention comprise a statin selected from the group consisting of atorvastatin, cerivastatin, fluvastatin, lovastatin, pitavastatin, pravastatin, rosuvastatin and simvastatin, whether in free form or as pharmaceutically acceptable salts and hydrates thereof, plus an antiflatulent agent. selected from the group consisting of simethicone and dimethicone. The combination of statins plus antiflatulent agents is useful in the prevention and management of flatulence caused by statins.

### FIELD OF THE INVENTION

The present invention relates to hypocholesterolemic compositions comprising statins plus antiflatulent agents.

### BACKGROUND OF THE INVENTION

Statins are inhibitors of hydroxymethylglutaryl-CoA reductase, a key enzyme in the synthesis of cholesterol, which directly lower cholesterol levels. These compounds are known to be safe and effective hypocholesterolemic agents and they, therefore, represent an important therapeutic contribution to the treatment of coronary heart disease and to the reduction of morbidity and mortality by such serious cardiovascular pathological conditions.

Statins commonly used in medicine are atorvastatin (U.S. Pat. No. 5,273,995), cerivastatin (U.S. Pat. No. 5,177,080), fluvastatin (U.S. Pat. No. 4,739,073), lovastatin (U.S. Pat. No. 4,231,938), pravastatin (U.S. Pat. No. 4,346,227), rosuvastatin (U.S. Pat. RE 37314) and simvastatin (U.S. Pat. No. 4,444,784). They may be used in free form or as pharmarceutically acceptable salts thereof, generally alkaline or alkaline-earth salts, whether anhydrous or hydrated; it is usually desirable to use the sodium or calcium salts. For example, in clinical practice, atorvastatin is used as sodium (2:1) trihydrate salt, cerivastatin, fluvastatin and pravastatin as sodium salt, rosuvastatin as sodium salt and lovastatin and simvastatin in free form. A more recent compound, pitavastatin (EP 304063), is currently under Phase III development in Europe.

WO 03/074034 describes pharmaceutical compositions with delayed release of anti-hypercholesterolemic drugs, e.g. statins. Stable tablets comprising simvastatiri are described in WO 03/086387.

Among the most significant and frequent side effects of statins is flatulence (Bakker-Arkema et al, Atherosclerosis 2000 March, 149(1), 123-9 [PubMed 10704623]; Black et al, Arch Intern Med 1998 March 23, 158(6), 577-84 [PubMed 9521221]; Posvar et al, J Clin Pharmacol 1996 August, 36(8), 728-31 [PubMed 8877677]; Boccuzzi et al, Am J Cardiol 1991 November 1, 68(11), 1127-31 [Pubmed 1951069]; Zeller et al, Drug Intell Clin Pharm 1988 July-August, 22(7-8), 542-5 [PubMed 3046888]), which may be the cause of discomfort and symptomatological confusion, since its symptoms may be like those of coronary heart disease which is the aim of hypolipemic therapy by statins.

Among substances capable of decreasing flatulence are the antiflatulent agents having an antifoaming action. Simethicone and dimethicone, for instance, are successfully applied to the management of flatulence and meteorism. These compounds are effective if appropriate sanitary/dietetic measures are further applied, for example, avoidance of carbonated drinks and flatulent food. Pharmaceutical compositions comprising an H₂ antagonist such as famotidine, an alginate and optionally an anti-flatulent amount of simethicone are, for instance, described in WO 95/01780.

A composition for forming a compressed solid dosage form that is a free-flowing admixture of simethicone, an adsorbant and optionally an active agent is described in EP-A 1297825.

Certain commercial products containing statins, like Lipitor (Atorvastatin as calcium 2:1 trihydrate), and formulations containing statins (WO 2004/021972) such as Pravastatin (WO 03/057195) have already been formulated with an antifoaming agent, i.e. an emulsion of simethicone However, the proportion of this compound is very low and it simply acts as a pharmaceutical carrier.

### OBJECT OF THE INVENTION

The object of the invention is to provide hypocholesterolemic compositions comprising a statin and an antiflatulent agent by antifoaming action in a proportion of active ingredient with the aim of relieving flatulence caused by the statin.

The combination of statins plus antiflatulent agents is useful in the prevention and management of flatulence caused by statins. This provides a better compliance of patients to the treatment and a better clinical understanding of symptoms, since both coronary heart diseases and flatulence are accompanied by thoracicoabdominal disturbances.

### SUMMARY OF THE INVENTION

The present invention relates to a pharmaceutical composition comprising a statin and an antiflatulent agent in a suitable proportion as active ingredient.

The compositions of the present invention comprise a statin preferably selected from the group consisting of atorvastatin, cerivastatin, fluvastatin, lovastatin, pitavastatin, pravastatin, rosuvastatin and simvastatin, whether in free form or as pharmaceutically acceptable salts and hydrates thereof, plus an antiflatulent agent preferably selected from the group consisting of simethicone and dimethicone.

The compositions of the present invention may be administered orally and are preferably in the form of solid or liquid compositions such as tablets, especially coated tablets, capsules, syrups, solutions, powders, granules, emulsions or the like.

The tablets and particularly the coated tablets are preferred.

The statins may be present in the tablets in an amount of 0.1 to 100 mg/tab. In turn, the antiflatulent agents may be present in the tablets in a proportion from 25 to 250 mg/tab.

The compositions of this invention further comprise other components selected from the group consisting of diluents, binders, disintegrants and lubricants, and mixtures thereof, which are commonly used in pharmaceutical technology. Other pharmaceutical excipients, like antioxidants and wetting agents, may be optionally added.

Due to the fact that statins are photosensitive it is convenient to protect the compositions, e.g. the tablets with a coating comprising cellulose or acrylic derivatives, as well as plasticizers and opacifiers. Optionally, it is possible to add different colouring agents.

### BRIEF DESCRIPTION OF THE DRAWING

FIG. 1 shows the in vitro dissolution profile, expressed in mean values, of the tablets of Example 4 comprising simvastatin plus simethicone, and other identical tablets without simethicone.

### DETAILED DESCRIPTION OF THE INVENTION

The present invention relates to a pharmaceutical composition comprising a statin and an antiflatulent agent in a suitable proportion as active ingredient.

An antiflatulent agent in a suitable proportion as active ingredient means an antiflatulent amount of said agent, i.e. an amount that effectively provides anti-flatulent relief. Likewise, the pharmaceutical compositions of the present invention comprise at least one statin in an amount that upon administration effectively provides a hypocholesterolemic effect.

According to one aspect of the present invention, the effective amount of the antiflatulant agent depends on the amount of statin. Thus, according to one embodiment, the weight ratio of antiflatulent agent versus statin is at least 0.25, preferably at least 0.50, 0.75 or 1.00, and in particular at least 1.25 or 1.50. Said ratios refer to the relative amounts to be administered or—since the statin(s) and the antiflatulant agent(s) are co-formulated—to the relative amounts that are present in the formulation. The maximum ratio of antiflatulent agent versus statin is not particularly limited. However, it may be expedient that the amount of antiflatulent agent does not exceed a certain proportion of the total weight of the formulation. Proportions of up to 50% by weight and especially of up to 30% by weight of the formulation may be expedient.

The compositions of the present invention comprise a statin preferably selected from the group consisting of atorvastatin, cerivastatin, fluvastatin, lovastatin, pitavastatin, pravastatin, rosuvastatin and simvastatin, whether in free form or as pharmaceutically acceptable salts and hydrates thereof, plus an antiflatulent agent preferably selected from the group consisting of simethicone and dimethicone.

Preferably, atorvastatin is used as calcium (2:1) trihydrate, cerivastatin, fluvastatin and pravastatin as sodium salt, rosuvastatin as calcium salt and lovastatin and simvastatin in free form.

The compositions of the present invention may be administered orally and are preferably in the form of solid compositions such as tablets, especially coated tablets, capsules, powder, granules or the like, or in the form of liquid compositions such as syrups, solutions, emulsions or the like. Solid compositions, especially tablets and particularly coated tablets are preferred. The ratios given above for the relative amounts of statin(s) versus antiflatulent agent(s) account for any one of these formulation types.

Statins may be present in the tablets in an amount of 0.1 to 100 mg/tablet. Thus, in case of a standard tablet weighing 400 mg, the proportion of statin may range from 0.025 to 25%. Usually, the amounts of statin per tablet may be 0.1, 2.5, 5, 10, 20, 40 and 80 mg. Therefore, for a standard tablet of 400 mg, the proportion of statin may be 0.025%, 0.625%, 1.25%, 2.5%, 5%, 10% and 20% respectively. Similar proportions apply to other compositions.

In turn, the antiflatulent agents may be present in the tablets in an amount of 25 to 250 mg/tablet. Therefore, for a standard tablet of 400 mg, the proportion of the antiflatulent agent may range from 6.25 to 62.5%. Similar proportions apply to other compositions which accordingly contain at least 6.25%, preferably more than 10% and especially more than 20% by weight of the composition.

Coated tablets comprise a core and a coating. In this case it is preferred that the core comprises both the statin(s) and the antiflatulent agent (s).

Preferred diluents in the tablets of the present invention are microcrystalline celluloses and derivatives thereof, for example Prosolv® which is a mixture of microcrystalline cellulose and colloidal silicon dioxide, lactose, mannitol, calcium phosphates, starch, and the like. Preferably, microcrystalline celluloses are Avicel® PH102 and Prosolv®.

Preferred binders in the tablets of the present invention are starch, polyethylene glycols, polyvinylpyrrolidone, cellulose derivatives, e.g., hydroxypropyl methylcellulose, and the like.

Preferred disintegrants in the tablets of the present invention are colloidal silicon dioxide, croscarmellose, polyvinylpyrrolidone, starch, and its pregelatinized derivatives, e.g., Primojel®, which is sodium starch glycolate, and the like. Preferably, Aerosil®, Acdisol® and polyvinylpyrrolidone are used.

Preferred lubricants in the tablets of the present invention are talc, magnesium stearate, stearic acid, sodium stearyl fumarate, high-molecular weight polyethylenglycol (4000-8000), e.g., PEG 8000, and the like. Preferably, sodium stearyl fumarate, talc and magnesium stearate are used.

Other pharmaceutic excipients, like antioxidants e.g., butylated hydroxyanisole, ascorbic acid or gluconolactone, and the like, and wetting agents e.g., sodium lauryl sulphate, and the like, may be optionally added.

The tablets of the present invention are preferably provided with a light-resistant coating. Preferably, the coating consists of a layer constituted by cellulose derivatives, for example, sodium hydroxypropyl methylcellulose (HPMC), acrylic polymers, plasticizers, for example, diethyl citrate, opacifiers, for example titanium dioxide, talc and stearic acid. They may optionally contain pigments for tablet-colouring. As pigments, ferric oxide derivatives are preferred.

The method for preparing the statin core with the antiflatulent agents may be by precompression, i.e., a previous compaction of the mixture, followed by sieving and final compression. They may also be obtained by wet granulation using a hydroalcohol solvent. These are standard procedures in pharmaceutical technology.

However, the applicants have discovered that the tablets of the present invention may be prepared advantageously by direct compression, i.e., by directly compressing all the components. Thus, simethicone, which is liquid, is incorporated in the form of an adsorbate with an adsorbent substance, for example, Prosolv®, mannitol, anhydrous colloidal silica (silicon dioxide) or lactose. It is then sieved and mixed with the other components to yield the final mixture. The procedure of direct compression is preferred rather than usual precompression procedures because of its lower cost and easier scale-up manufacturing.

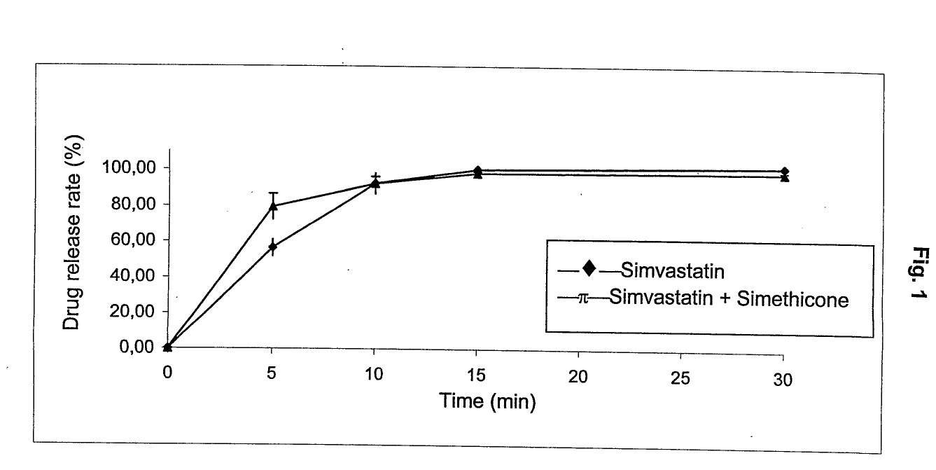

The solubility of the tablets of the present invention is not affected by the presence of an antiflatulent agent. Thus, FIG. 1 shows that the dissolution profiles of the new tablets of Example 4, which contain the antiflatulent agent, namely simethicone, are not different from those of conventional tablets without antiflatulent agent. Consequently, there are no significant differences in the pharmaceutical behaviour of either preparation, in such a way that treatment of patients taking statins may be easily replaced with the tablets of the present invention.

The present invention is further illustrated by—but not limited to—the following examples.

### EXAMPLE 1

400 mg tablet containing 40 mg of atorvastatin (calcium trihydrate) and 115 mg of simethicone

| | | |
|---|---|---|
| Atorvastatin (calcium trihydrate) | 40 | mg |
| Simethicone | 115 | mg |
| Anhydrous colloidal silica | 10 | mg |
| Sodium croscarmellose | 10 | mg |
| Sodium stearyl fumarate | 15 | mg |
| Microcrystalline cellulose Avicel PH102 | q.s. 400 | mg |

### EXAMPLE 2

400 mg tablet containing 20 mg of simvastatin and 125 mg of simethicone

| | | |
|---|---|---|
| Simvastatin | 20 | mg |
| Simethicone | 125 | mg |
| Polyvinylpyrrolidone | 15 | mg |
| Sodium croscarmellose | 5 | mg |
| Sodium stearyl fumarate | 15 | mg |
| Sodium lauryl sulfate | 4 | mg |
| Butylated hydroxyanisole | 5 | mg |
| Lactose | q.s. 400 | mg |

### EXAMPLE 3

400 mg tablet containing 10 mg of sodium pravastatin and 125 mg of simethicone

| | | |
|---|---|---|
| Sodium pravastatin | 10 | mg |
| Simethicone | 125 | mg |
| Primojel ® | 20 | mg |
| Talc | 12 | mg |
| Magnesium stearate | 4 | mg |
| Prosolv ® | q.s. 400 | mg |

### EXAMPLE 4

400 mg tablet containing 40 mg of simvastatin and 125 mg of simethicone

| | | |
|---|---|---|
| Simvastatin | 40 | mg |
| Simethicone | 125 | mg |
| Primojel ® | 16 | mg |
| Silicon dioxide | 43 | mg |
| Talc | 12 | mg |
| Magnesium stearate | 4 | mg |
| Lactose (direct compression) | q.s. 400 | mg |

1. A pharmaceutical composition comprising a statin and an antiflatulent agent in a suitable proportion as active ingredient.

2. A pharmaceutical composition according to claim 1 wherein the statin is selected from the group consisting of atorvastatin, cerivastatin, fluvastatin, lovastatin, pitavastatin, pravastatin, rosuvastatin and simvastatin, and pharmaceutically acceptable salts and hydrates thereof.

3. A pharmaceutical composition according to claim 2 wherein the statin is simvastatin or a pharmaceutically acceptable salt thereof.

4. A pharmaceutical composition according to any one of claims 1 to 3 wherein the antiflatulent agent is selected from the group consisting of simethicone and dimethicone.

5. A pharmaceutical composition according to claim 4 wherein the antiflatulent agent is simethicone.

6. A pharmaceutical composition according to claim 1 wherein the composition is a tablet, capsule, syrup, solution, powder, granule, or emulsion.

7. A pharmaceutical composition according to claim 6 wherein the tablet is a coated tablet.

8. A pharmaceutical composition according to claim 7 wherein the coated tablet comprises a core and a coating, the core comprising the statin and the antiflatulent agent.

9. A pharmaceutical composition according to claim 1, wherein the weight ratio of antiflatulent agent versus statin is at least 0.25.

10. A pharmaceutical composition according to claim 1, wherein the weight ratio of antiflatulent agent versus statin is at least 1.50.

11. A pharmaceutical composition according to claim 6 or 7 wherein simvastatin is present in an amount from 2.5 to 100 mg per tablet.

12. A pharmaceutical composition according to claim 11 wherein simvastatin is present in an amount from 5 to 80 mg per tablet.

13. A pharmaceutical composition according to claims 6 or 7 wherein simethicone is present in an amount from 25 to 250 mg per tablet.

14. A pharmaceutical composition according to claim 13 wherein simethicone is present in an amount of 125 mg per tablet.

15. A pharmaceutical composition according to claim 1 further comprising one or more diluents, one or more binders, one or more disintegrants and one or more lubricants.

16. A pharmaceutical composition according to claim 15 wherein the diluent is selected from the group consisting of microcrystalline celluloses and their derivatives, lactose, mannitol, calcium phosphates, starch, and the mixtures thereof.

17. A pharmaceutical composition according to claim 15 wherein the binder is selected from the group consisting of starch, polyethylene glycols, polyvinylpyrrolidones, cellulose derivatives, and the mixtures thereof.

18. A pharmaceutical composition according to claim 15 wherein the disintegrant is selected from the group consisting of colloidal silicon dioxide, croscarmellose, 1a polyvinylpyrrolidone, starch and its pregelatinized derivatives, and the mixtures thereof.

19. A pharmaceutical composition according to claim 15 wherein the lubricant is selected from the group consisting of talc, magnesium stearate, stearic acid, sodium stearyl fumarate, PEG 8000, and the mixtures thereof.

20. A pharmaceutical composition according to claim 1 further comprising one or more antioxidants and one or more wetting agents.

21. A pharmaceutical composition according to claim 7 wherein the coating of the tablet comprises a cellulose derivative or its pharmaceutically.

22. A pharmaceutical composition according to claim 21 wherein the cellulose derivative is hydroxypropyl methylcellulose.

23. A pharmaceutical composition according to claim 1 further comprising one or more colouring agents.

24. A process for preparing a pharmaceutical composition according to claim 1 by direct compression of components thereof.
